(19) United States
(12) Patent Application Publication
(10) Pub. No.: US 2021/0070948 A1
(43) Pub. Date: Mar. 11, 2021

(54) A Method for Producing High-activity Lignin and By-product Furfural and Application

(71) Applicant: JINAN SHENGQUAN GROUP SHARE-HOLDING CO., LTD., Jinan (CN)

(72) Inventors: Chengzhen JIANG, Jinan (CN); Shaofeng GAO, Jinan (CN); Feng SHI, Jinan (CN)

(73) Assignee: JINAN SHENGQUAN GROUP SHARE-HOLDING CO., LTD., Jinan (CN)

(21) Appl. No.: 16/772,170

(22) PCT Filed: Nov. 23, 2018
(86) PCT No.: PCT/CN2018/117273

§ 371 Date: Jun. 12, 2020

(30) Foreign Application Priority Data
Dec. 13, 2017 (CN) 201711328316.3

(51) Int. Cl. C08H 7/00; C07D 307/50; C08G 16/02

(52) U.S. Cl. CPC C08H 6/00; C08G 16/0262; C07D 307/50

(57) ABSTRACT

A method for producing high-activity lignin and by-product furfural and an application, includes: using a dissolving pulp pre-hydrolysate and/or sulfite cooking liquor as the raw material, and catalyzing by employing an acid as a catalyst to obtain the lignin and furfural, wherein the acid is one or a combination of at least two of sulfuric acid, phosphoric acid, nitric acid, hydrochloric acid, and acetic acid. According to the method, the yield of the obtained lignin and furfural is further increased by catalyzing the dissolving pulp pre-hydrolysate and/or the sulfite cooking liquor with the acid. Lignin especially has higher reactivity and low molecular weight, and the ratio of 5-methyl furfural in furfural to furfural is in a proper range, so that the strength of furan resin prepared from furfural is improved.

### CROSS-REFERENCE TO THE RELATED APPLICATIONS

This application is the national phase entry of International Application No. PCT/CN2018/117273, filed on Nov. 23, 2018, which is based upon and claims priority to Chinese Patent Application No. 201711328316.3, filed on Dec. 13, 2017, the entire contents of which are incorporated herein by reference.

### TECHNICAL FIELD

This invention belongs to the technical field of biomass, and relates to a method for producing high-activity lignin and by-product furfural and application thereof.

### BACKGROUND

As an important organic chemical raw material and chemical solvent, furfural is widely used in the industries such as petroleum, chemicals, medicine, food, synthetic rubber and synthetic resin, and the global demand for furfural is huge every year. It can selectively extract unsaturated components from petroleum and vegetable oils, and can also extract aromatic components from lubricants and diesel oil. With the intensification of the energy crisis, the use of renewable agricultural and forestry wastes to produce high value-added furfural, and the development and utilization of its downstream chemical products have received increasing attention. China's furfural takes up a significant position in the world furfural trade, and has made great progress in recent years.

The production of furfural is based on the chemical conversion of pentose. At present, on the industrial scale, the only way to obtain furfural is still hydrolysis with corn cobs as the raw material. According to the principle of hydrolysis and the process of generating furfural, the methods for preparing furfural can be divided into direct method and indirect method. The direct method (one-step method) is to put the hemicellulose-containing raw materials into the hydrolysis pot, and under the action of catalyst and heat, the hemicellulose is hydrolyzed to pentose, and the pentose is dehydrated to generate furfural. The indirect method (two-step method) includes two steps, namely, hydrolysis reaction of hemicellulose and dehydration reaction of pentose, which are completed in different kinds of equipment.

CN 103261184 A discloses a method for preparing furfural from lignocellulose feedstock containing dextran and xylan, which makes the feedstock contact water in the presence of an acid catalyst, and then makes the obtained mixture contact at least one water-immiscible organic solvent to form a mixture containing an aqueous phase and an organic phase, and furfural is prepared under suitable reaction conditions, and preferably distributed into the organic phase from which furfural can be recovered.

CN 104072450 A discloses a method for preparing 5-hydroxymethyl furfural and furfural from biomass raw materials, comprising: adding biomass raw materials, solvent, solid phosphate catalyst and soluble inorganic salt to a reactor to form a reaction system. Under the protection of inert gas within the temperature range of 20-400° C., carry out dehydration reaction for 0-100 h to obtain 5-hydroxymethyl furfural and furfural products with a higher yield.

None of the above patents can achieve the joint production of lignin and furfural, and the furfural extraction by two-phase extraction has a low yield and a complicated process. It is of important research significance to further improve the yield of furfural and achieve the joint production of lignin at the same time.

In the production process of pre-hydrolyzed sulfate dissolving pulp, the first step is pre-hydrolysis, which is carried out under acidic conditions. Generally, hemicellulose undergoes acidic hydrolysis and dissolves under the condition of acetic acid produced by steam high temperature self-hydrolysis. In this way, the content of hemicellulose (especially pentosan) in the raw material is greatly reduced. While hemicellulose undergoes acid hydrolysis, part of the lignin also dissolves under acidic conditions.

At present, after most of the pre-hydrolysate is concentrated by dissolving pulp production enterprises, it is added to an alkali recovery boiler for incineration, which not only wastes steam energy, but also increases the load of the alkali recovery boiler, thereby increasing the production cost of the dissolving pulp. The main components in the pre-hydrolysate are pentosan and lignin. It is of great significance to further use it comprehensively, turn waste into treasure, increase the capacity of the existing equipment of the dissolving pulp production enterprises, and reduce the production cost of dissolving pulp.

### SUMMARY

In view of the said shortcomings in the prior art, the object of this invention is to provide a method for producing high-activity lignin and by-product furfural and an application. The said method further increases the yield of the obtained lignin and furfural by catalyzing the dissolving pulp pre-hydrolysate and/or the sulfite cooking liquor with acid uses acid to catalyze dissolving pulp pre-hydrolysate and/or sulfite cooking liquor to obtain the lignin and furfural, in particular, the ratio of 5-methyl furfural in furfural to furfural is in a proper range, so that the strength of furan resin prepared from furfural can be further improved.

To achieve the above object, this invention adopts the following technical solutions:

In the first aspect, this invention provides a method for producing lignin and by-product furfural. The said method comprises: using dissolving pulp pre-hydrolysate and/or sulfite cooking liquor as the raw material, and catalyzing by using acid as a catalyst to obtain the lignin and furfural;

Wherein, the said acid is one or a mixture of at least two of sulfuric acid, phosphoric acid, nitric acid, hydrochloric acid, and acetic acid.

In this invention, dissolving pulp pre-hydrolysate and/or sulfite cooking liquid are used as specific raw materials, and acid is used for catalysis for joint production of lignin and furfural, which improves the yield of lignin and furfural.

The following are preferred technical solutions of this invention, but not as limitations to the technical solutions provided by this invention. Through the following preferred technical solutions, the technical objects and beneficial effects of this invention can be better achieved.

According to this invention, the said dissolving pulp pre-hydrolysate and/or sulfite cooking liquor contains lignin and pentosan.

According to this invention, the content of the said lignin is 1-60%, for example, it can be 1%, 2%, 3%, 5%, 6%, 8%, 10%, 12%, 15%, 16%, 18%, 20%, 22%, 23%, 25%, 26%, 28%, 30%, 32%, 35%, 36%, 38%, 40%, 42%, 45%, 48%, 50%, 52%, 55%, 56%, 58% or 60%, preferably 1-30%, further preferably 1-10%.

According to this invention, the content of the said pentosan is 1-60%, for example, it can be 1%, 2%, 3%, 5%, 6%, 8%, 10%, 12%, 15%, 16%, 18%, 20%, 22%, 23%, 25%, 26%, 28%, 30%, 32%, 35%, 36%, 38%, 40%, 42%, 45%, 48%, 50%, 52%, 55%, 56%, 58% or 60%, preferably 1-30%, further preferably 1-10%.

According to this invention, the said acid is a mixture of two or more of sulfuric acid, phosphoric acid, nitric acid, hydrochloric acid, and acetic acid, preferably a mixture of sulfuric acid and phosphoric acid.

In this invention, the inventors unexpectedly discovered that compared to the use of one single acid for catalysis, the content of 5-methyl furfural in the produced furfural accounts for about 5% of the said furfural by using mixed acid for catalysis, so that the strength of furan resin prepared from such furfural is further improved, which is conducive to the subsequent comprehensive utilization of furfural.

According to this invention, the said mixed acid is a mixture of sulfuric acid and phosphoric acid, and the volume ratio of sulfuric acid to phosphoric acid is 1:(0.5-3), for example, it can be 1:0.5, 1:0.8, 1:1, 1:1.2, 1:1.5, 1:1.8, 1:2, 1:2.2, 1:2.5, 1:2.8 or 1:3, preferably 1:(0.8-1.5).

In this invention, the inventors found that the mixture of sulfuric acid and phosphoric acid has the best catalytic effect, especially when the volume ratio of sulfuric acid to phosphoric acid is in the range of 1:(0.5-3), and the furan resin has the highest strength when it is prepared from furfural obtained with such volume ratio within the range of 1:(0.8-1.5).

According to this invention, the method for producing lignin and by-product furfural includes the following steps:

(1) Concentrate the dissolving pulp pre-hydrolysate and/or sulfite cooking solution, add it to the heating medium, add acid, then mix and stir, react under suitable conditions, and distill to obtain furfural aqueous solution;

(2) Make the remaining feed liquid in step (1) further react, and precipitate and filter to obtain lignin.

According to this invention, the solid content after concentration in step (1) is 8-40%, for example, it can be 8%, 10%, 12%, 13%, 15%, 18%, 20%, 22%, 23%, 25%, 26%, 28%, 30%, 32%, 35%, 38% or 40%, preferably 10-30%.

In this invention, the inventors found that when the solid content after concentration is 8-40%, especially 10-30%, the obtained lignin has better activity and the yield of furfural is higher.

According to this invention, the heating medium described in step (1) can dissolve lignin, and the said heating medium is a liquid and/or solid with a boiling point or melting point greater than 160° C., preferably a liquid and/or solid with a boiling point or melting point greater than 180° C.

Preferably, the said liquid heating medium in step (1) is any one or a mixture of at least two of dimethyl sulfoxide, dimethyl sulfone, diethyl sulfone, diphenyl sulfone, gamma-valerolactone, polyethylene glycol, glycerin, 1,3-propylene glycol, sulfolane, isophorone and propylene carbonate.

Preferably, the solid heating medium in step (1) is any one or a mixture of at least two of carbon powder, granular salt, silica or rock powder.

According to this invention, the added amount of the said acid accounts for 0.1-10% of the mass of the said reaction liquid, for example, it can be 0.1%, 0.2%, 0.3%, 0.5%, 0.6%, 0.8%, 1%, 1.2%, 1.3%, 1.5%, 1.6%, 1.8%, 2%, 2.3%, 2.5%, 2.8%, 3%, 3.2%, 3.5%, 3.8%, 4%, 4.2%, 4.5%, 4.8%, 5%, 5.5%, 6%, 6.5%, 7%, 7.5%, 8%, 8.5%, 9%, 9.5% or 10%, preferably 0.15-5%.

According to this invention, the said stirring rate is 50-300 rpm, for example, it can be 50 rpm, 80 rpm, 100 rpm, 120 rpm, 130 rpm, 150 rpm, 160 rpm, 180 rpm, 200 rpm, 220 rpm, 250 rpm, 260 rpm, 280 rpm or 300 rpm, preferably 100-200 rpm.

According to this invention, the pressure of the said reaction in step (1) is −0.1-0.5 Mpa. For example, it can be −0.1 Mpa, 0 Mpa, 0.1 Mpa, 0.2 Mpa, 0.3 Mpa, 0.4 Mpa, or 0.5 Mpa, preferably −0.1-0.3 Mpa.

According to this invention, the temperature of the said reaction in step (1) is 160-200° C., for example, it can be 160° C., 162° C., 165° C., 168° C., 170° C., 172° C., 175° C., 178° C., 180° C., 182° C., 185° C., 190° C., 192° C., 195° C. or 200° C., preferably 170-190° C.

According to this invention, it is acceptable as long as the flow rate for adding the feedstock to the heating medium in step (1) can control the moisture content in the reaction system not more than 15%, preferable the moisture content in the reaction system not more than 10%, more preferably the moisture content in the reaction system not more than 5%, which is not particularly limited here, and those skilled in the art can select an appropriate flow rate according to needs.

According to this invention, the time of the said reaction in step (2) is less than 120 min, preferably not more than 90 min, further preferably 40-90 min.

In this invention, the said reaction time is the residence and reaction time after the raw material enters the heating medium, and the reaction time shall be controlled within 120 min, especially 90 min is optimal; if the reaction time is more than 120 min, lignin will be subject to partial carbonization and condensation, thus affecting the subsequent use effect of lignin.

According to this invention, the pressure of the said reaction in step (2) is −0.1-0.5 Mpa, for example, it can be −0.1 Mpa, 0 Mpa, 0.1 Mpa, 0.2 Mpa, 0.3 Mpa, 0.4 Mpa or 0.5 Mpa, preferably-0.1-0.3 Mpa.

According to this invention, the temperature of the said reaction in step (2) is 160-200° C., for example, it can be 160° C., 162° C., 65° C., 68° C., 170° C., 172° C., 175° C., 178° C., 180° C., 182° C., 185° C., 190° C., 192° C., 195° C. or 200° C., preferably 170-180° C.

According to this invention, the recovery of the lignin in step (2) can also be achieved through dissolving with alkaline solution, and the said alkali is any one or a mixture of at least two of sodium hydroxide, potassium hydroxide or calcium hydroxide.

As a preferred technical solution, the said method for producing lignin and by-product furfural includes the following steps:

(1) Concentrate the dissolving pulp pre-hydrolysate and/or sulfite cooking solution to 10-30% solid content, add it to the heating medium, add acid that accounts for 0.1-2% of the mass of the reaction liquid, then mix and stir at a stirring rate of 50-300 rpm, and react at a pressure of −0.1-0.5 Mpa and a temperature of 160-200° C., and distill to obtain furfural aqueous solution;

(2) Make the remaining liquid in step (1) further react at a pressure of −0.1-0.5 Mpa and a temperature of 160-200° C. for less than 120 minutes, and then precipitate and filter to obtain lignin.

In a second aspect, this invention provides furfural and lignin prepared with the method described in the first aspect.

According to this invention, the said furfural includes a mixture of furfural and 5-methyl furfural.

According to this invention, the mass ratio of furfural to 5-methyl furfural is (16-20):1, for example, it can be 16:1, 17:1, 18:1, 19:1 or 20:1.

In a third aspect, this invention provides a lignin as described in the second aspect for preparing any one or a combination of at least two of furan resin, phenolic resin, dye dispersant, cement water reducing agent, carbon black binder, refractory binder or metal smelting binder.

In a fourth aspect, this invention provides a furfural as described in the second aspect for preparing furan resin.

Compared with the prior art, this invention has the following beneficial effects:

(1) According to the method of this invention, dissolving pulp pre-hydrolysate and/or sulfite cooking liquid is used as the raw material, and mixed acid is used as a catalyst for catalysis and joint production of lignin and furfural. Any two of sulfuric acid, phosphoric acid, nitric acid, hydrochloric acid and acetic acid are used as mixed acid for catalysis to further promote the reaction and formation of the product, which is conducive to improving the yield of furfural and lignin. The furfural resin prepared from such furfural has high strength and the prepared lignin has good activity.

(2) The furfural prepared with this invention can be used to prepare furan resin. Compared with furan resin prepared directly from furfural or a mixture of furfural and 5-methyl furfural, such furan resin has increased bonding strength, high strength, good stability at high temperatures, and very broad application prospects;

(3) The lignin prepared with the invention has good activity, and can be further used for preparing furan resin or phenol resin, etc., which is conducive to the subsequent comprehensive utilization of lignin.

### DETAILED DESCRIPTION OF THE EMBODIMENTS

The technical terms mentioned in this specification have the same meanings as those generally understood by those skilled in the art. In case of inconsistency, the definitions in this specification shall prevail.

In this specification, the dissolving pulp pre-hydrolysate refers to the liquid containing pentosan, lignin, inorganic substances and other substances obtained through pre-hydrolyzing plant raw materials in the first step in the production process of dissolving pulp with the “pre-hydrolytic sulfate process”.

In this specification, sulfite cooking liquid refers to the liquid containing lignosulfonates, carbohydrates and their degradation products, inorganic substances and other substances (commonly known as “red liquid”) obtained through cooking the plant raw materials with sulfite in the production process of papermaking or dissolving pulp with the “sulfite process”.

In this specification, pentosan, also known as pentose, refers to the general term of polymers composed of five-carbon sugars in hemicellulose, and various plant raw materials contain varying amounts of pentosan.

In this specification, heating medium refers to a carrier of heat. In industry, a heat transfer medium that transfers heat energy from a heating device to a heat-using unit in an indirect manner is called heating medium.

The technical solutions of this invention are further described below through specific embodiments.

This invention will be further described in detail in combination with the following embodiments. The embodiments described below are only preferred ones of this invention, and are not intended to limit this invention in other forms. Any person skilled in the art may use the technical content disclosed above to change to equivalent embodiments with equivalent changes.

Any simple modifications and equivalent changes made to the following embodiments that depart from the solutions of this invention but are based on the technical essence of this invention shall fall within the protection scope of this invention.

In the embodiments, the dissolving pulp pre-hydrolysate comes from Sappi, South Africa.

### Embodiment 1—Preparation of Lignin and Furfural (I)

The method for joint production of lignin and furfural specifically includes the following steps:

(1) Concentrate the dissolving pulp pre-hydrolysate to the reaction liquid with a solid content of 15%, wherein the pentosan content is 9.39%, the lignin content is 5.42%, and the ash content is 0.19%;

(2) Add 100 g isophorone to the 250 ml high-pressure reactor, stir at a rate of 100 rppm, heat to 180° C., then add sulfuric acid catalyst that accounts for 0.8% of the mass of the reaction liquid to the reaction liquid, keep the temperature unchanged at 180° C., add the reaction liquid to the reactor at a rate of 10 g/min, and distill out the distillate at the same time, control the distillation valve to make the reactor pressure at 0.2 Mpa, maintain the moisture content in the reaction liquid within 5%, distill and react for 40 min, stop feeding, collect distillate and weigh a total of 140 g, which is the said furfural aqueous solution;

(3) Make the reaction mixture further react at a pressure of 0.2 Mpa and a temperature of 180° C. for 20 min, cool to room temperature, add water to precipitate and filter, wash multiple times, and the brownish yellow solid obtained after recovering isophorone is lignin.

### Embodiment 2—Preparation of Lignin and Furfural (II)

The method for joint production of lignin and furfural specifically includes the following steps:

(1) Concentrate the dissolving pulp pre-hydrolysate to the reaction liquid with a solid content of 20%, wherein the pentosan content is 12.48%, the lignin content is 7.21%, and the ash content is 0.31%;

(2) Add 100 g sulfolane to the 250 ml high-pressure reactor, stir at a rate of 150 rppm, heat to 170° C., then add phosphoric acid catalyst that accounts for 1.0% of the mass of the reaction liquid to the reaction liquid, keep the temperature unchanged at 170° C., add the reaction liquid to the reactor at a rate of 10 g/min, and distill out the distillate at the same time, control the distillation valve to make the reactor pressure at 0.3 Mpa, maintain the moisture content in the reaction liquid within 3%, distill and react for 40 min, stop feeding, collect distillate and weigh a total of 132 g, which is the said furfural aqueous solution;

(3) Make the reaction mixture further react at a pressure of 0.3 Mpa and a temperature of 170° C. for 20 min, cool to room temperature, add water to precipitate and filter, wash multiple times, and the brownish yellow solid obtained after recovering sulfolane is lignin.

### Embodiment 3—Preparation of Lignin and Furfural (III)

The method for joint production of lignin and furfural specifically includes the following steps:

(1) Concentrate the dissolving pulp pre-hydrolysate to the reaction liquid with a solid content of 8%, wherein the pentosan content is 4.99%, the lignin content is 2.88%, and the ash content is 0.13%;

(2) Add 100 g gamma-valerolactone to the 250 ml four-necked bottle, stir at a rate of 200 rppm, vacuum the four-necked bottle to −0.1 Mpa, heat to 200° C., then add hydrochloric acid catalyst that accounts for 0.1% of the mass of the reaction liquid to the reaction liquid, keep the temperature unchanged at 200° C., add the reaction liquid to the four-necked bottle at a rate of 10 g/min, and obtain the distillate through vacuum distillation at the same time, make the pressure of the four-necked bottle at −0.1 Mpa, maintain the moisture content in the reaction liquid within 5%, distill and react for 90 min, stop feeding, collect distillate and weigh a total of 126 g, which is the said furfural aqueous solution;

(3) Make the reaction mixture further react at a pressure of −0.1 Mpa and a temperature of 200° C. for 30 min, cool to room temperature, add water to precipitate and filter, wash multiple times, and the brownish yellow solid obtained after recovering gamma-valerolactone is lignin.

### Embodiment 4—Preparation of Lignin and Furfural (IV)

The method for joint production of lignin and furfural specifically includes the following steps:

(1) Concentrate the sulfite cooking liquid to the reaction liquid with a solid content of 40%, wherein the pentosan content is 12.48%, the lignin content is 7.21%, and the ash content is 0.31%;

(2) Add 100 g propylene carbonate to the 250 ml high-pressure reactor, stir at a rate of 50 rppm, heat to 160° C., then add acetic acid catalyst that accounts for 9.6% of the mass of the reaction liquid to the reaction liquid, keep the temperature unchanged at 160° C., add the reaction liquid to the reactor at a rate of 10 g/min, and distill out the distillate at the same time, control the distillation valve to make the reactor pressure at 0.5 Mpa, maintain the moisture content in the reaction liquid within 5%, distill and react for 40 min, stop feeding, collect distillate and weigh a total of 162 g, which is the said furfural aqueous solution;

(3) Make the reaction mixture further react at a pressure of 0.5 Mpa and a temperature of 160° C. for 30 min, cool to room temperature, add water to precipitate and filter, wash multiple times, and the brownish yellow solid obtained after recovering propylene carbonate is lignin.

### Embodiment 5—Preparation of Lignin and Furfural (V)

Compared with Embodiment 1, the acid in step (2) is a combination of sulfuric acid and hydrochloric acid, wherein the volume ratio of sulfuric acid to hydrochloric acid is 1:0.5, the added mixed acid accounts for 0.15% of the mass of the said reaction liquid, and the other components and steps are the same as those in Embodiment 1.

### Embodiment 6—Preparation of Lignin and Furfural (VI)

Compared with Embodiment 1, the acid in step (2) is a combination of phosphoric acid and nitric acid, wherein the volume ratio of phosphoric acid to nitric acid is 1:3, the added mixed acid accounts for 0.8% of the mass of the said reaction liquid, and the other components and steps are the same as those in Embodiment 1.

### Embodiment 7—Preparation of Lignin and Furfural (VII)

Compared with Embodiment 1, the acid in step (2) is a combination of sulfuric acid and acetic acid, wherein the volume ratio of sulfuric acid to acetic acid is 1:1, the added mixed acid accounts for 0.8% of the mass of the said reaction liquid, and the other components and steps are the same as those in Embodiment 1.

### Embodiment 8—Preparation of Lignin and Furfural (VII)

Compared with Embodiment 1, the acid in step (2) is a combination of sulfuric acid and phosphoric acid, wherein the volume ratio of sulfuric acid to acetic acid is 1:1.2, the added mixed acid accounts for 0.8% of the mass of the said reaction liquid, and the other components and steps are the same as those in Embodiment 1.

### Comparative Example 1

Compared with Embodiment 1, the raw material in step (1) is corn cobs, and the other components and steps are the same as those in Embodiment 1.

### Comparative Example 2

Compared with Embodiment 1, the dissolving slurry pre-hydrolysate in step (1) is concentrated to the reaction liquid with a solid content of 5%, and the other components and steps are the same as those in Embodiment 1.

### Comparative Example 3

Compared with Embodiment 1, the dissolving slurry pre-hydrolysate in step (1) is concentrated to the reaction liquid with a solid content of 43%, and the other components and steps are the same as those in Embodiment 1.

### Comparative Example 4

Compared with Embodiment 1, the amount of acid added in step (2) accounts for 12% of the mass of the reaction liquid, and the other components and steps are the same as those in Embodiment 1.

Test and Analysis

The components of the lignin and furfural prepared in Embodiments 1-8 and Comparative Examples 1-4 were analyzed with liquid chromatography, and their physical and chemical properties were also analyzed. The results are shown in Table 1:

TABLE 1

| | | | Average | | | | | |
|---|---|---|---|---|---|---|---|---|
| | | | ethanol | | Phenol | Weight | Furfural | |
| | Lignin | Ash | dissolution | Softening | hydroxyl | average | | 5-methyl |
| | yield | content | rate | point | content | molecular | Furfural | furfural |
| | (%) | (%) | (%) | (° C.) | (mol) | weight | (%) | (%) |
| Embodiment | 13 | 4.21 | 82 | 76-78 | 4.86 | 1256 | 96.8 | 3.2 |
| 1 | | | | | | | | |
| Embodiment | 12 | 4.13 | 85 | 80-82 | 4.75 | 1247 | 96.4 | 3.6 |
| 2 | | | | | | | | |
| Embodiment | 16 | 2.33 | 87 | 85-87 | 4.7 | 1376 | 96.1 | 3.9 |
| 3 | | | | | | | | |
| Embodiment | 19 | 3.52 | 88 | 83-85 | 4.9 | 1305 | 96.0 | 4.0 |
| 4 | | | | | | | | |
| Embodiment | 28 | 1.54 | 90 | 76-78 | 5.04 | 1204 | 95.3 | 4.7 |
| 5 | | | | | | | | |
| Embodiment | 26 | 1.87 | 89 | 74-76 | 5.23 | 1186 | 95.5 | 4.5 |
| 6 | | | | | | | | |
| Embodiment | 29 | 1.22 | 88 | 67-69 | 5.16 | 1240 | 95.2 | 4.8 |
| 7 | | | | | | | | |
| Embodiment | 34 | 0.88 | 91 | 72-74 | 5.67 | 1140 | 94.6 | 5.4 |
| 8 | | | | | | | | |
| Comparative | 3 | 8.45 | 70 | 95-97 | 3.12 | 1845 | 99.1 | 0.9 |
| Example 1 | | | | | | | | |
| Comparative | 8 | 5.12 | 75 | 88-90 | 4.05 | 1564 | 97.7 | 2.3 |
| Example 2 | | | | | | | | |
| Comparative | 7 | 5.04 | 76 | 87-89 | 4.16 | 1579 | 97.9 | 2.1 |
| Example 3 | | | | | | | | |
| Comparative | 10 | 7.89 | 78 | 85-87 | 4.24 | 1642 | 98.4 | 1.6 |
| Example 4 | | | | | | | | |

As can be seen from Table 1, in Embodiments 1-8, the yield of lignin from the furfural obtained through catalysis with the mixed acid can reach 34%, the ash content is low, the content of impurities is low, the average ethanol dissolution rate is high, the softening point is low, the content of phenolic hydroxyl group is large, and the weight average molecular weight is low. According to the comparison between Comparative Examples 1-4 and Embodiment 1, the lignin from the furfural prepared from other raw materials (such as corn cobs) is reduced, the ash content is high, the content of impurities is high, and the weight average molecular weight is high; moreover, the proportion of 5-methyl furfural in furfural is low. The concentration of solid content is not within the scope of this application or if the acid catalyst added is excessive, the yield of lignin from the prepared furfural will be reduced, the ash content will be high, the content of impurities will be high, the weight average molecular weight will be high, and the proportion of 5-methyl furfural in furfural will be low.

### Embodiment 9—Synthesis of Furan Resin (I)

The synthesis method of the said furan resin includes the following steps:

(1) Take 900 g furfural in Embodiment 1 and add it to the reaction kettle, add copper catalyst to the said reaction kettle for hydrogenation reaction, filter the catalyst, and distill furfuryl alcohol, and 900 g furfuryl alcohol is obtained;

(2) Add 176 g formaldehyde (concentration: 37%) to the reaction kettle, start stirring, add 80 kg urea, adjust the pH value with alkaline solution to 8.8-9.0 after the urea is dissolved, raise the temperature to 90±2° C. and react for 1 hour; add the said furfuryl alcohol of 200 g and further react for 1 hour; adjust the pH value of the material system to 4.5-4.8, and react at a reaction temperature of 95±2° C. for 1.5 hours;

(3) Add 20 g urea, adjust the pH value of the material system to 7.8-8.0, react for 1 hour at a temperature of 75±2° C., cool the material system and stop the reaction; dehydrate 110 g under vacuum, then add the said furfuryl alcohol of about 623 g, stir evenly, discharge the material, and the obtained product is about 989 g.

### Embodiment 10—Synthesis of Furan Resin (II)

Compared with Embodiment 9, except that furfural in Embodiment 2 is used to prepare the said furan resin, the other components and steps are the same as those in Embodiment 9.

### Embodiment 11—Synthesis of Furan Resin (III)

Compared with Embodiment 9, except that furfural in Embodiment 3 is used to prepare the said furan resin, the other components and steps are the same as those in Embodiment 9.

### Embodiment 12—Synthesis of Furan Resin (IV)

Compared with Embodiment 9, except that furfural in Embodiment 4 is used to prepare the said furan resin, the other components and steps are the same as those in Embodiment 9.

### Embodiment 13—Synthesis of Furan Resin (V)

Compared with Embodiment 9, except that furfural in Embodiment 5 is used to prepare the said furan resin, the other components and steps are the same as those in Embodiment 9.

### Embodiment 14—Synthesis of Furan Resin (VI)

Compared with Embodiment 9, except that furfural in Embodiment 6 is used to prepare the said furan resin, the other components and steps are the same as those in Embodiment 9.

### Embodiment 15—Synthesis of Furan Resin (VII)

Compared with Embodiment 9, except that furfural in Embodiment 7 is used to prepare the said furan resin, the other components and steps are the same as those in Embodiment 9.

### Embodiment 16—Synthesis of Furan Resin (VIII)

Compared with Embodiment 9, except that furfural in Embodiment 8 is used to prepare the said furan resin, the other components and steps are the same as those in Embodiment 9.

### Comparative Example 5

Compared with Embodiment 9, except that furfural in Comparative Example 1 is used to prepare the said furan resin, the other components and steps are the same as those in Embodiment 9.

### Comparative Example 6

Compared with Embodiment 9, except that furfural in Comparative Example 2 is used to prepare the said furan resin, the other components and steps are the same as those in Embodiment 9.

### Comparative Example 7

Compared with Embodiment 9, except that furfural in Comparative Example 3 is used to prepare the said furan resin, the other components and steps are the same as those in Embodiment 9.

### Comparative Example 8

Compared with Embodiment 9, except that furfural in Comparative Example 4 is used to prepare the said furan resin, the other components and steps are the same as those in Embodiment 9.

### Comparative Example 9

Compared with Embodiment 9, except that furfural available commercially is used to prepare the said furan resin, the other components and steps are the same as those in Embodiment 9.

Test and Analysis

Physical and chemical test was carried out for the furan resins prepared in Embodiments 9-16 and Comparative Examples 5-9, and the analysis method of nitrogen content and free formaldehyde was carried out in accordance with JBT7526-2008 Self-set Furan Resin for Foundry.

The test results are shown in Table 2:

TABLE 2

| | | | | | Nitrogen |
|---|---|---|---|---|---|
| | pH | Density | Viscosity | Free aldehyde | content |
| Embodiment 9 | 8.24 | 1.18 | 22.01 | 0.78 | 4.80 |
| Embodiment 10 | 8.23 | 1.18 | 22.12 | 0.78 | 4.81 |
| Embodiment 11 | 8.23 | 1.17 | 22.:4 | 0.78 | 4.80 |
| Embodiment 12 | 8.26 | 1.17 | 22.15 | 0.78 | 4.76 |
| Embodiment 13 | 8.25 | 1.18 | 21.81 | 0.78 | 4.75 |
| Embodiment 14 | 8.24 | 1.19 | 21.83 | 0.78 | 4.76 |
| Embodiment 15 | 8.23 | 1.19 | 21.72 | 0.78 | 4.75 |
| Embodiment 16 | 8.22 | 1.19 | 21.65 | 0.78 | 4.73 |
| Comparative | 8.21 | 1.17 | 22.33 | 0.78 | 4.82 |
| example 5 | | | | | |
| Comparative | 8.21 | 1.18 | 22.34 | 0.78 | 4.81 |
| example 6 | | | | | |
| Comparative | 8.23 | 1.17 | 22.37 | 0.78 | 7.80 |
| example 7 | | | | | |
| Comparative | 8.22 | 1.18 | 22.35 | 0.78 | 4.82 |
| example 8 | | | | | |
| Comparative | 8.21 | 1.16 | 22.45 | 0.78 | 4.83 |
| example 9 | | | | | |

As can be seen from Table 2, the viscosity of the furan resins prepared in Embodiments 9-16 is lower than that in Comparative Examples 5-9, and there are no obvious differences in other physical and chemical properties.

A sand mixing test was carried out for the furan resins prepared in Embodiments 9-16 and Comparative Examples 5-9 to test the tensile strength. The method for testing the tensile strength of resin bonded sand at room temperature was carried out according to JBT 7526-2008 Self-set Furan Resin for Foundry.

The specific conditions of the first sand mixing test are as follows: room temperature: 12.1° C., humidity: 50.1%, resin addition amount: 1.0%, curing agent GC09 addition amount: 50%, the specific conditions of the second sand mixing test are as follows: room temperature: 8.2° C., humidity: 22.4%, resin addition amount: 1.0%, curing agent GC09 addition amount: 50%; the test results are shown in Table 3:

TABLE 3

| | The first tensile strength | | | The second tensile strength | | |
|---|---|---|---|---|---|---|
| | (standard sand) | | | (standard sand) | | |
| | 50 min | 4 h | 24 h | 30 min | 4 h | 24 h |
| Embodiment 9 | 0.28 | 2.25 | 2.54 | 0.26 | 1.12 | 1.62 |
| Embodiment 10 | 0.27 | 2.28 | 2.49 | 0.25 | 1.11 | 1.59 |
| Embodiment 11 | 0.27 | 2.19 | 2.41 | 0.22 | 1.07 | 1.51 |
| Embodiment 12 | 0.26 | 2.16 | 2.37 | 0.23 | 1.06 | 1.46 |
| Embodiment 13 | 0.30 | 2.54 | 2.54 | 0.28 | 1.18 | 1.68 |
| Embodiment 14 | 0.31 | 2.53 | 2.62 | 0.28 | 1.19 | 1.71 |
| Embodiment 15 | 0.32 | 2.67 | 2.79 | 0.27 | 1.17 | 1.69 |
| Embodiment 16 | 0.35 | 2.87 | 2.98 | 0.30 | 1.23 | 1.75 |
| Comparative | 0.21 | 1.35 | 1.78 | 0.20 | 0.89 | 1.23 |
| example 5 | | | | | | |
| Comparative | 0.25 | 1.86 | 1.92 | 0.22 | 0.95 | 1.38 |
| example 6 | | | | | | |
| Comparative | 0.24 | 1.81 | 1.91 | 0.27 | 0.96 | 1.34 |
| example 7 | | | | | | |
| Comparative | 0.23 | 1.72 | 1.89 | 0.30 | 0.99 | 1.29 |
| example 8 | | | | | | |
| Comparative | 0.22 | 1.3 | 1.75 | 0.20 | 0. 90 | 1.22 |
| example 9 | | | | | | |

As can be seen from Table 3, the furfural obtained in this invention contains 5-methyl furfural, and the furfuryl alcohol after hydrogenation contains 5-methyl furfuryl alcohol. From the comparison of Embodiments 9-16, it can be seen that the strength of the furan resin prepared from furfural catalyzed with a mixed acid is higher than that of the furan resin prepared from furfural catalyzed with a single acid; according to the comparison between Comparative Examples 5-9 and Embodiment 9, the strength of the furan resin prepared with other raw materials (such as corn cobs) or commercially available furfural is obviously reduced, and the concentration of solid content is not within the scope of this application. The strength of the prepared furan resin is also significantly reduced. Excessive acid catalyst added will also affect the strength of the prepared furan resin; it can be seen that with respect to the tensile strength of synthetic furan resin, the tensile strength of furan resin made from furfural prepared in this invention is 20% higher than that of furan resin made from commercially available furfural.

In summary, the furfural prepared with this invention can be used to prepare furan resin. Compared with furan resin prepared directly from furfural or a mixture of furfural and 5-methyl furfural, such furan resin has increased bonding strength, high strength, good stability at high temperatures, and very broad application prospects.

The applicants declare that the said embodiments are the preferred ones of this invention, which are not intended to limit the embodiments of this invention. Any other changes, modifications, replacements, combinations and simplifications made without departing from the spirit and principle of this invention shall be equivalent substitutions, and are all included in the protection scope of this invention. That does not mean that this invention must rely on the said detailed methods for implementation. Those skilled in the art shall understand that any improvement to this invention, equivalent replacement of various raw materials of the product in this invention, addition of auxiliary components, selection of specific methods and so on shall fall within the scope of protection and disclosure of this invention.

What is claimed is:

1. A method for producing a composition comprising lignin and by-product furfural, wherein the method comprises: using a dissolving pulp pre-hydrolysate and/or sulfite cooking liquor as a raw material, and catalyzing by using an acid as a catalyst to obtain the lignin and furfural;
wherein, the acid is one or a mixture of at least two of sulfuric acid, phosphoric acid, nitric acid, hydrochloric acid, and acetic acid.

2. The method according to claim 1, wherein the dissolving pulp pre-hydrolysate and/or sulfite cooking liquor contains the lignin and pentosan;
a content of the lignin is 1-60% or 1-30% or 1-10%;
a content of the pentosan is 1-60% or 1-30% or 1-10%;
the acid is the mixture of the at least two of sulfuric acid, phosphoric acid, nitric acid, hydrochloric acid, and acetic acid or the acid is a mixture of sulfuric acid and phosphoric acid;
a volume ratio of sulfuric acid to phosphoric acid is 1:(0.5-3) or 1:(0.8-1.5).

3. The method according to claim 1, comprising the following steps:
(1) concentrate the dissolving pulp pre-hydrolysate and/or sulfite cooking solution, add a concentrated dissolving pulp pre-hydrolysate and/or sulfite cooking solution to a heating medium, add the acid, then mix and stir, react under suitable conditions, and distill to obtain a furfural aqueous solution;
(2) make a remaining feed liquid in step (1) further react, and precipitate and filter to obtain the lignin.

4. The method according to claim 3, wherein a solid content after concentration in step (1) is 1-60% or 8-40% or 10-30%;
the heating medium described in step (1) is a liquid and/or solid with a boiling point or melting point greater than 160° C. or a liquid and/or solid with the boiling point or melting point greater than 180° C.;
the liquid heating medium in step (1) is any one or a mixture of at least two of dimethyl sulfoxide, dimethyl sulfone, diethyl sulfone, diphenyl sulfone, gamma-valerolactone, polyethylene glycol, glycerin, 1,3-propylene glycol, sulfolane, isophorone and propylene carbonate;
the heating medium in step (1) is any one or a mixture of at least two of carbon powder, granular salt, silica or rock powder.

5. The method according to claim 4, wherein an added amount of the acid accounts for 0.1-10% of a mass of a reaction liquid or the added amount of the acid accounts for 0.15-5%;
a stirring rate is 50-300 rpm or 100-200 rpm;
a pressure of a reaction in step (1) is −0.1-0.5 Mpa or −0.1-0.3 Mpa;
a temperature of the reaction in step (1) is 160-200° C. or 170-190° C.

6. The method according to claim 5, wherein a time of a reaction in step (2) is less than 120 min or the time of the reaction in step (2) is not more than 90 min or the time of the reaction in step (2) is 40-90 min;
a pressure of the reaction in step (2) is −0.1-0.5 Mpa or −0.1-0.3 Mpa;
a temperature of the reaction in step (2) is 160-200° C. or 170-180° C.

7. The method according to claim 6, comprising the following steps:
(1) concentrate the dissolving pulp pre-hydrolysate and/or sulfite cooking solution to 10-30% solid content, add a concentrated dissolving pulp pre-hydrolysate and/or sulfite cooking solution to the heating medium, add the acid that accounts for 0.1-2% of the mass of the reaction liquid, then mix and stir at the stirring rate of 50-300 rpm, and react at a pressure of −0.1-0.5 Mpa and a temperature of 160-200° C., and distill to obtain the furfural aqueous solution;
(2) make the remaining feed liquid in step (1) further react at the pressure of −0.1-0.5 Mpa and the temperature of 160-200° C. for less than 120 minutes, and then precipitate and filter to obtain the lignin.

8. A composition comprising lignin and furfural, prepared according to the method of claim 1.

9. The composition according to claim 8, wherein the furfural comprises a mixture of the furfural and 5-methyl furfural;
a mass ratio of the furfural to 5-methyl furfural is (16-20):1.

10. The composition according to claim 8, wherein the lignin is used for preparing any one or a combination of at least two of furan resin, phenolic resin, dye dispersant, cement water reducing agent, carbon black binder, refractory binder or metal smelting binder;
the furfural is used to prepare furan resin after hydrogenation.

11. The method according to claim 2, comprising the following steps:
(1) concentrate the dissolving pulp pre-hydrolysate and/or sulfite cooking solution, add a concentrated dissolving pulp pre-hydrolysate and/or sulfite cooking solution to a heating medium, add the acid, then mix and stir, react under suitable conditions, and distill to obtain a furfural aqueous solution;
(2) make a remaining feed liquid in step (1) further react, and precipitate and filter to obtain the lignin.

12. The method according to claim 1, wherein a solid content after concentration in step (1) is 1-60% or 8-40% or 10-30%;
the heating medium described in step (1) is a liquid and/or solid with a boiling point or melting point greater than 160° C. or a liquid and/or solid with the boiling point or melting point greater than 180° C.;
the liquid heating medium in step (1) is any one or a mixture of at least two of dimethyl sulfoxide, dimethyl sulfone, diethyl sulfone, diphenyl sulfone, gamma-valerolactone, polyethylene glycol, glycerin, 1,3-propylene glycol, sulfolane, isophorone and propylene carbonate;
the heating medium in step (1) is any one or a mixture of at least two of carbon powder, granular salt, silica or rock powder.

13. The method according to claim 2, wherein a solid content after concentration in step (1) is 1-60% or 8-40% or 10-30%;
the heating medium described in step (1) is a liquid and/or solid with a boiling point or melting point greater than 160° C. or a liquid and/or solid with the boiling point or melting point greater than 180° C.;
the liquid heating medium in step (1) is any one or a mixture of at least two of dimethyl sulfoxide, dimethyl sulfone, diethyl sulfone, diphenyl sulfone, gamma-valerolactone, polyethylene glycol, glycerin, 1,3-propylene glycol, sulfolane, isophorone and propylene carbonate;
the heating medium in step (1) is any one or a mixture of at least two of carbon powder, granular salt, silica or rock powder.

14. The method according to claim 1, wherein an added amount of the acid accounts for 0.1-10% of a mass of a reaction liquid or the added amount of the acid accounts for 0.15-5%;
a stirring rate is 50-300 rpm or 100-200 rpm;
a pressure of a reaction in step (1) is −0.1-0.5 Mpa or −0.1-0.3 Mpa;
a temperature of the reaction in step (1) is 160-200° C. or 170-190° C.

15. The method according to claim 2, wherein an added amount of the acid accounts for 0.1-10% of a mass of a reaction liquid or the added amount of the acid accounts for 0.15-5%;
a stirring rate is 50-300 rpm or 100-200 rpm;
a pressure of a reaction in step (1) is −0.1-0.5 Mpa or −0.1-0.3 Mpa;
a temperature of the reaction in step (1) is 160-200° C. or 170-190° C.

16. The method according to claim 3, wherein an added amount of the acid accounts for 0.1-10% of a mass of a reaction liquid or the added amount of the acid accounts for 0.15-5%;
a stirring rate is 50-300 rpm or 100-200 rpm;
a pressure of a reaction in step (1) is −0.1-0.5 Mpa or −0.1-0.3 Mpa;
a temperature of the reaction in step (1) is 160-200° C. or 170-190° C.

17. The method according to claim 1, wherein a time of a reaction in step (2) is less than 120 min or the time of the reaction in step (2) is not more than 90 min or the time of the reaction in step (2) is 40-90 min;
a pressure of the reaction in step (2) is −0.1-0.5 Mpa or −0.1-0.3 Mpa;
a temperature of the reaction in step (2) is 160-200° C. or 170-180° C.

18. The method according to claim 2, wherein a time of a reaction in step (2) is less than 120 min or the time of the reaction in step (2) is not more than 90 min or the time of the reaction in step (2) is 40-90 min;
a pressure of the reaction in step (2) is −0.1-0.5 Mpa or −0.1-0.3 Mpa;
a temperature of the reaction in step (2) is 160-200° C. or 170-180° C.

19. The method according to claim 3, wherein a time of a reaction in step (2) is less than 120 min or the time of the reaction in step (2) is not more than 90 min or the time of the reaction in step (2) is 40-90 min;
a pressure of the reaction in step (2) is −0.1-0.5 Mpa or −0.1-0.3 Mpa;
a temperature of the reaction in step (2) is 160-200° C. or 170-180° C.

20. The method according to claim 1, comprising the following steps:
(1) concentrate the dissolving pulp pre-hydrolysate and/or sulfite cooking solution to 10-30% solid content, add a concentrated dissolving pulp pre-hydrolysate and/or sulfite cooking solution to the heating medium, add the acid that accounts for 0.1-2% of the mass of the reaction liquid, then mix and stir at the stirring rate of 50-300 rpm, and react at a pressure of −0.1-0.5 Mpa and a temperature of 160-200° C., and distill to obtain the furfural aqueous solution;
(2) make the remaining feed liquid in step (1) further react at the pressure of −0.1-0.5 Mpa and the temperature of 160-200° C. for less than 120 minutes, and then precipitate and filter to obtain the lignin.
